(19) United States
(12) Patent Application Publication
(10) Pub. No.: US 2004/0236190 A1
(43) Pub. Date: Nov. 25, 2004

(54) Network based patient monitor apparatus

(75) Inventors: Chao-Fa Lee, Taipei City (TW); Cheng-Hsing Kuo, Taipei City (TW); Ya-Ting Lu, Taipei City (TW); Jen-Chien Lin, Taipei City (TW); Shu-Ting Liao, Taipei City (TW)

Correspondence Address: BACON & THOMAS, PLLC, 625 SLATERS LANE, FOURTH FLOOR, ALEXANDRIA, VA, 22314

(73) Assignee: Tatung Co., Ltd., Taipei City (TW)

(21) Appl. No.: 10/847,401

(22) Filed: May 18, 2004

(30) Foreign Application Priority Data
May 19, 2003 (TW) 092209136

(51) Int. Cl. A61B 5/00

(52) U.S. Cl. 600/300

(57) ABSTRACT

A patient monitor apparatus coupled to LAN of a hospital is provided. The apparatus comprises a body signal reading module for obtaining at least one body signal, a signal processing module for digitally processing the body signal to obtain at least one record of body data prior to converting into at least one Web page, and a monitor module for showing data, and a network interface interconnected the signal processing module and the LAN for sending the Web page to any computer over the LAN. A medical staff (e.g., nurse or doctor) whether locally or remotely located, can immediately know the conditions of a patient by typing a unique address of the apparatus on a computer by viewing the Web pages of the patient.

### BACKGROUND OF THE INVENTION

1. Field of the Invention

The present invention relates to a patient monitor apparatus and, more particularly, to a network based patient monitor apparatus.

2. Description of Related Art

Many medical systems of hospitals are operated by means of computers and even networks due to technological advancements of information industry. It is common that LAN is typically incorporated in a modern hospital for coupling clinics, wards, and nurse stations together. Also, a monitor apparatus is installed in a specific ward or clinic. As such, an additional database or network has to be established in order to retrieve data from the monitor apparatus. This data retrieval is cumbersome.

Moreover, the cost for the hospital is much higher if a monitor apparatus is installed in each one of a plurality of wards and/or clinics. Further, an additional network has to be established. This in turn can increase cost. Thus, prior technique is not cost effective. Therefore, it is desirable to take advantage of the existing LAN of a hospital for establishing novel network based patient monitor apparatuses so that a medical staff, for example, a nurse or doctor whether locally or remotely located, can immediately know the conditions of a patient by using a computer to retrieve the patient's data from an associated monitor apparatus.

### SUMMARY OF THE INVENTION

An object of the present invention is to provide a network based patient monitor apparatus installed in a hospital so that for example, a nurse or doctor whether locally or remotely located, can immediately know the conditions of a patient by using a computer to retrieve the patient's data from an associated monitor apparatus over the LAN.

Another object of the present invention is to provide a network based patient monitor apparatus, so that the installation of a great number of fixed monitor apparatuses as experienced by the prior art can be eliminated so as to reduce cost significantly.

To achieve the above and other objects, the present invention provides a patient monitor apparatus coupled to LAN of a hospital, the monitor apparatus having a unique address so that one of at least one terminal coupled to the LAN is adapted to retrieve data from the patient monitor apparatus by using the address, comprising a body signal reading module for obtaining at least one body signal; a signal processing module for digitally processing the at least one body signal to obtain at least one record of body data, converting the at least one record of body data into at least one Web page, and storing the Web pages in a memory; a monitor module for receiving the at least one record of body data from the signal processing module and displaying the same, the monitor module including at least one input means for data input so that the signal processing module is adapted to process input data and the at least one body signal for obtaining at least one record of body data, and a network interface coupled to the signal processing module so that the signal processing module is adapted to send the at least one Web page to the at least one terminal over the LAN.

Other objects, advantages, and novel features of the present invention will become more apparent from the detailed description when taken in conjunction with the accompanying drawings.

### BRIEF DESCRIPTION OF THE DRAWINGS

FIG. 1 is a block diagram of the present invention; and

FIG. 2 schematically depicts the connection of a preferred embodiment of the present invention.

### DETAILED DESCRIPTION OF THE PREFERRED EMBODIMENT

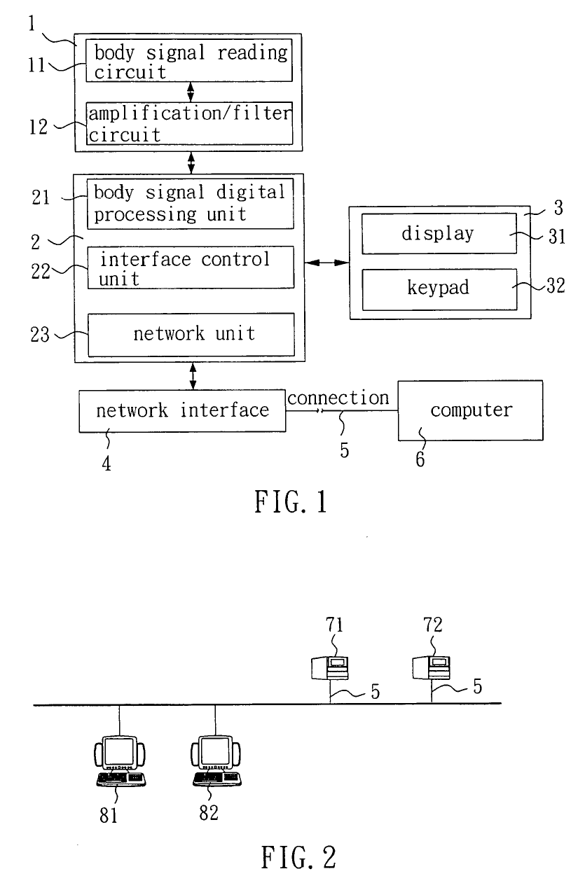

The network based patient monitor apparatus of the present invention is implemented in a hospital. Specifically, the monitor apparatus is mounted on a moveable platform or cart so that for example, a nurse or doctor can push the platform or cart to any clinic or ward for measuring body conditions of a patient. With reference to FIG. 1, there is shown a block diagram of the present invention. The present invention comprises a body signal reading module 1, a signal processing module 2, a monitor module 3, and a network interface 4. The body signal reading module 1 comprises a body signal reading circuit 11 and an amplification/filter circuit 12. The signal processing module 2 comprises a body signal digital processing unit 21, an interface control unit 22, and a network unit 23. The monitor module 3 comprises a display 31 and a keypad 32. Each component will be described in detail below.

The body signal reading circuit 11 is adapted to read at least one body signal (e.g., blood pressure, oxygen content in the blood, electrocardiogram, etc.) of a patient by means of an external sensor or instrument. The amplification/filter circuit 12 is adapted to receive the at least one body signal not being processed from the body signal reading circuit 11 and amplify and filter the same. For example, DC (direct current) gain, AC (alternating current) gain, high pass filter, or low pass filter is performed on the at least one body signal so as to obtain a processed body signal.

The body signal digital processing unit 21 is adapted to receive the processed at least one body signal from the body signal reading module 1 and digitally process the same so as to obtain at least one record of body data corresponding to the at least one digital body signal. And in turn, the at least one record of body data is sent to the monitor module 3 for showing the same on the display 31. A user may either input patients' data (e.g., patient numbers or bar codes of patients) or control a processing mode of the signal processing module 2 by means of the keypad 32 of the monitor module 3. For the latter case, a control message is generated by the keypad 32 and the control message is immediately sent to the signal processing module 2. As such, the interface control unit 22 is able to control a processing mode of the signal processing module 2. Eventually, the signal processing module 2 is adapted to process at least one body signal based on the control message.

In response to obtaining at least one body signal at the signal processing module 2, the signal processing module 2 converts the same into one or more Web pages. In the embodiment, the Web page is preferably written in the format of HTTP (Hyper Text Transfer Protocol). Next, the signal processing module 2 either stores the Web pages in a memory (not shown) or sends the Web pages to the network unit 23 for storage. In the embodiment, the network unit 23 is preferably a Web server and comprises an installed TCP/IP (Transmission Control Protocol/Internet Protocol) based software. As an end, the network unit 23 is able to place the Web pages on the LAN.

In the embodiment, the network interface 4 is preferably an Ethernet interface coupled to the signal processing module 2. As such, the signal processing module 2 is adapted to send the Web pages to any computer 6 coupled to the LAN through the network interface 4 and the connection 5.

With reference to FIG. 2, the connection of a preferred embodiment of the present invention is illustrated. As shown, a plurality of monitor apparatuses 71 and 72 and a plurality of computers 81 and 82 are coupled together by the connection 5 of the LAN. Each one of the monitor apparatuses 71 and 72 has a unique address so that for example, a user (e.g., doctor or nurse) can input the address of the monitor apparatus 71 on either computer 81 or 82 to view the Web pages of patients in order to see updated data of patients.

In brief, the present invention can convert measured body signal into Web pages which are in turn placed on the LAN of a hospital. As such, a medical staff (e.g., nurse or doctor) whether locally or remotely located, can immediately know the conditions of a patient by using a computer coupled to a specific monitor apparatus over the LAN by viewing the Web pages of the patient. Further, the present invention can reduce cost significantly.

Although the present invention has been explained in relation to its preferred embodiment, it is to be understood that many other possible modifications and variations can be made without departing from the spirit and scope of the present invention as hereinafter claimed.

What is claimed is:

1. A patient monitor apparatus coupled to LAN of a hospital, the monitor apparatus having a unique address so that one of at least one terminal coupled to the LAN is adapted to retrieve data from the patient monitor apparatus by using the address, comprising:
a body signal reading module for obtaining at least one body signal;
a signal processing module for digitally processing the at least one body signal to obtain at least one record of body data, converting the at least one record of body data into at least one Web page, and storing the Web pages in a memory;
a monitor module for receiving the at least one record of body data from the signal processing module and displaying the same, the monitor module including at least one input means for data input so that the signal processing module is adapted to process input data and the at least one body signal for obtaining at least one record of body data, and
a network interface coupled to the signal processing module so that the signal processing module is adapted to send the at least one Web page to the at least one terminal over the LAN.

2. The patient monitor apparatus as claimed in claim 1, wherein the body signal reading module comprises a body signal reading circuit for reading the at least one body signal, and an amplification/filter circuit for receiving the at least one body signal from the body signal reading circuit and amplifying and filtering the same for obtaining at least one processed body signal.

3. The patient monitor apparatus as claimed in claim 1, wherein each of the at least one input means is adapted to input a control message in the signal processing module so that the signal processing module is adapted to process the at least one body signal based on the control message.

4. The patient monitor apparatus as claimed in claim 3, wherein the signal processing module comprises a body signal digital processing unit for digitally processing the at least one body signal, and an interface control unit for receiving the control message so as to process the at least one body signal based on the control message.

5. The patient monitor apparatus as claimed in claim 1, wherein the signal processing module further comprises a network unit for processing and converting the at least one record of body data into at least one Web page based on a protocol.

6. The patient monitor apparatus as claimed in claim 5, wherein the protocol is a TCP/IP.

7. The patient monitor apparatus as claimed in claim 5, wherein each of the at least one terminal is adapted to retrieve data on the at least one Web page from the patient monitor apparatus by using the address.

8. The patient monitor apparatus as claimed in claim 1, wherein the network interface is coupled to the LAN via a connection.

9. The patient monitor apparatus as claimed in claim 1, wherein each of the at least one input means is a keypad.
